(19) United States
(12) Patent Application Publication
(10) Pub. No.: US 2022/0238189 A1
(43) Pub. Date: Jul. 28, 2022

(54) METHOD OF EXTRACTING DRUG INFORMATION BASED ON BIOACTIVITY DATA, METHOD OF CONSTRUCTING DRUG SCREENING LIBRARY, AND ANALYSIS APPARATUS

(71) Applicant: KAIPHARM CO., LTD., Seoul (KR)

(72) Inventors: Wan Kyu KIM, Seoul (KR); Se Ra PARK, Seoul (KR); Yea Jee KWON, Seoul (KR)

(73) Assignee: KAIPHARM CO., LTD., Seoul (KR)

(21) Appl. No.: 17/409,331

(22) Filed: Aug. 23, 2021

(30) Foreign Application Priority Data
Jan. 25, 2021 (KR) 10-2021-0010022

(51) Int. Cl. G16C 20/30; G16C 20/40

(52) U.S. Cl. CPC G16C 20/30; G16C 20/40

(57) ABSTRACT

A method of discovery a drug based on bioactivity data includes extracting, by the analysis apparatus, bioassay data from a bioassay database, classifying, by the analysis apparatus, a plurality of candidate compounds included in the bioassay data into a similar compound group and a dissimilar compound group based on similarity with the target compound, calculating, by the analysis apparatus, a relative activity score (RAS) based on activity information on compounds belonging to the similar compound group and the dissimilar compound group; and selecting, by the analysis apparatus, at least some of the plurality of candidate compounds included in the bioassay data as a drug candidate substance based on the RAS.

### CROSS-REFERENCE TO RELATED APPLICATION

This application claims priority under 35 U.S.C. § 119(a) to Korean Patent Application No. 10-2021-0010022 (filed on Jan. 25, 2021), which is hereby incorporated by reference in its entirety.

### BACKGROUND

The following description relate to an in silico drug screening technique.

New drug development industries consume a great deal of time and money. Recently, an in silico drug discovery technique based on artificial intelligence or big data analysis is attracting attention. Traditional in silico drug discovery techniques are mainly based on structural analysis of drugs and target proteins. The in silico drug discovery techniques mainly utilize bioassay data to test diverse range of compounds' activities extracted from published or unpublished experimental results. The bioassay data is composed of accumulated data of experiments. The bioassay data may not include data of specific compounds. Hence, there is a need for a in silico drug discovery technique with the specific compounds.

### SUMMARY

In one general aspect, there is provided a method of extracting drug information based on bioactivity data including receiving, by an analysis apparatus, information on a target compound, extracting, by the analysis apparatus, bioassay data from a bioassay database, classifying, by the analysis apparatus, a plurality of candidate compounds included in the bioassay data into a similar compound group and a dissimilar compound group based on similarity with the target compound, calculating, by the analysis apparatus, a relative activity score (RAS) based on activity information on compounds belonging to the similar compound group and the dissimilar compound group, and selecting, by the analysis apparatus, at least some of the plurality of candidate compounds included in the bioassay data as an analysis target based on the RAS.

In another aspect, there is provided a method of constructing a drug discovery library based on bioactivity data including receiving, by an analysis apparatus, information on a target compound, extracting, by the analysis apparatus, bioassay data from a bioassay database, classifying, by the analysis apparatus, a plurality of candidate compounds included in the bioassay data into a similar compound group and a dissimilar compound group based on similarity with the target compound, calculating, by the analysis apparatus, a relative activity score (RAS) based on activity information on whether each of the compounds belonging to the similar compound group and the dissimilar compound group and a target protein are activated, and selecting, by the analysis apparatus, the bioassay data as library data for drug research when the RAS is greater than or equal to a threshold value.

In yet another aspect, there is provided an analysis apparatus for discovery a drug based on bioactivity data includes an input device configured to receive information on a target compound, a communication device configured to receive specific bioassay data from a bioassay database, a storage device configured to store an instruction for discovery drug candidate substances based on structural information and activity information of compounds, and an processor configured to evaluate similarity between candidate compounds included in the bioassay data and the target compound, classify the candidate compounds into a similar compound group and a dissimilar compound group based on the similarity, calculate a relative activity score (RAS) based on activity information on the compounds belonging to the similar compound group and the dissimilar compound group, and select at least some of the candidate compounds as a drug candidate substance based on the RAS.

### BRIEF DESCRIPTION OF THE DRAWINGS

FIG. 1 illustrating an example of a system for extracting drug information using bioassay data;

FIG. 2 illustrating an example of a flowchart of a process of extracting drug information based on bioactivity data;

FIG. 3 illustrating another example of the flowchart of the process of extracting drug information based on bioactivity data;

FIG. 4 illustrating an example of a process of determining compound similarity;

FIG. 5 illustrating an example of an analysis apparatus for discovery a drug based on bioactivity data;

FIG. 6 is an example of experimental results according to the present embodiment; and

FIG. 7 is another example of the experimental results according to the present embodiment.

Throughout the drawings and the detailed description, the same reference numerals refer to the same elements. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences of operations described herein are merely examples, and are not limited to those set forth herein, but may be changed as will be apparent after an understanding of the disclosure of this application, with the exception of operations necessarily occurring in a certain order. Also, descriptions of features that are known in the art may be omitted for increased clarity and conciseness.

The features described herein may be embodied in different forms, and are not to be construed as being limited to the examples described herein. Rather, the examples described herein have been provided merely to illustrate some of the many possible ways of implementing the methods, apparatuses, and/or systems described herein that will be apparent after an understanding of the disclosure of this application.

As used herein, the term “and/or” includes any one and any combination of any two or more of the associated listed items.

The terminology used herein is for describing various examples only, and is not to be used to limit the disclosure. The articles “a,” “an,” and “the” are intended to include the plural forms as well, unless the context clearly indicates otherwise. The terms “comprises,” “includes,” and “has” specify the presence of stated features, numbers, operations, members, elements, and/or combinations thereof, but do not preclude the presence or addition of one or more other features, numbers, operations, members, elements, and/or combinations thereof.

Terms to be given below will be described.

An analysis apparatus uses compound information and bioassay data, which are digital information, to extract desired specific drug-related information. The analysis apparatus may access a public database through a network and extract bioassay data that includes experiment results by other researchers. The analysis apparatus is a device capable of processing data and may be formed as a personal computer (PC), a smart device, a server, or the like.

The compound information may include various pieces of information on compounds. The compound information may include structures, functions, physicochemical properties, and the like of the corresponding compound. A format representing the compound structure may be any one of various types. For example, the compound structure may be represented in any one of types such as a molfile (MOL), a structure-data file (SDF), a simplified molecular-input line-entry system (SMILES), and an international chemical identifier (InChI). The compound information may further include an identifier for identifying the compound.

The bioassay data refers to data on the results of the known bioassay experiment that has already been performed. The bioassay data includes a plurality of compound sets. Accordingly, the analysis apparatus may extract all pieces of bioassay data for a specific compound based on an identifier of a specific compound. Also, the analysis apparatus may identify all compound sets including the specific compound among specific bioassay data.

Hit compounds refer to a compound having a reaction to a specific target, a specific protein, or a specific biological activity. Here, the reaction may be binding promotion, binding inhibition or phenotypic measurements. The hit compound may be represented by various indicators. It is assumed that the bioassay data includes quantified information on activity of a specific compound against a specific target or a specific biological activity. Accordingly, the analysis apparatus may determine whether the compound is active or inactive based on evaluation criteria (for example, set hit compound score)) for the activity.

A chemical fingerprint refers to bit vector data representing a plurality of structural characteristics of any compound. The fingerprint may be represented in various types or formats. For example, extended connectivity fingerprint (ECFP) corresponds to one of chemical fingerprints as a circular topological fingerprint representing a chemical structure of a compound.

Hit enriched bioassays refer to bioassays by which users may identify hypothetically similar compounds of their desired compounds. The analysis apparatus may determine whether bioassay data is significant based on a specific threshold value.

The bioassay data may not include information on all compounds since the bioassay data corresponds to cumulative experimental results. As a result, the bioassay data may have no or little information on a specific compound. The technology described below is to provide a drug discovery technique that may be utilized even when there are no experimental results for the specific compound in the bioassay data.

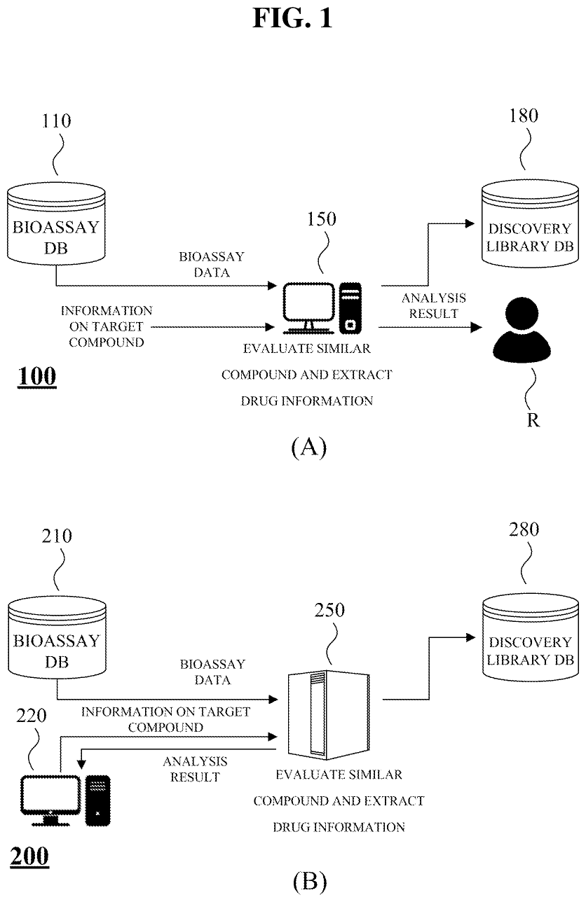

FIG. 1 shows examples of a system for extracting drug information using bioassay data. The analysis apparatus may be implemented in various forms such as a computer device, a PC, a smart device, and a server on a network. FIG. 1 illustrates examples in which the analysis apparatus is a computer terminal 150 and a server 250.

FIG. 1, (A) is a system 100 in which a user (researcher) performs drug discovery using the computer terminal 150. The bioassay database (DB) 110 stores bioassay data. The bioassay DB 110 may be a DB such as the PubChem. The bioassay DB 110 may be connected to the computer terminal 150 through a wired or wireless network. Alternatively, the bioassay DB 110 may be a storage medium physically directly connected to the computer terminal 150. The bioassay data may include the information on the candidate compounds and the experimental results. The candidate compound refers to a compound that may be a candidate for a specific drug.

The computer terminal 150 receives information on a target compound from a user. The information on the target compound may include an identifier of the target compound. The computer terminal 150 extracts bioassay data from the bioassay DB 110. The computer terminal 150 may evaluate the similarity between the candidate compound and the target compound included in the bioassay data based on the installed programs. The computer terminal 150 may calculate predetermined scores using the candidate compounds classified depending on whether the candidate compounds are similar to the target compound and the activity information of the candidate compounds. The computer terminal 150 may extract the specific drug candidate or the drug-related information based on the score for the candidate compound. The computer terminal 150 may construct a discovery library DB 180 that stores specific drug-related information based on the analysis results. The computer terminal 150 may provide the analysis results to a user.

FIG. 1, (B) is a system 200 in which a user accesses the analysis server 250 through a user terminal 220 to perform the drug discovery. The bioassay DB 210 stores the bioassay data. The bioassay DB 210 may be the DB such as the PubChem. The bioassay DB 210 may be connected to the analysis server 250 through the wired or wireless network. Alternatively, the bioassay DB 210 may be a storage medium physically directly connected to the analysis server 250. The bioassay data may include the information on the candidate compounds and the experimental results. The candidate compound refers to a compound that may be a candidate for a specific drug.

The user terminal 220 receives the information on the target compound from a user. The information on the target compound may include the identifier of the target compound. The analysis server 250 receives the information on the target compound from the user terminal 220. The analysis server 250 extracts the bioassay data from the bioassay DB 210. The analysis server 250 may evaluate the similarity between the candidate compound included in the bioassay data and the target compound based on the installed programs. The analysis server 250 may calculate predetermined scores using candidate compounds classified depending on whether the candidate compounds are similar to the target compound and activity information of the candidate compounds. The analysis server 250 may extract a specific drug candidate or drug-related information based on the score for the candidate compound. The analysis server 250 may construct a discovery library DB 280 that stores the specific drug-related information based on the analysis results. The analysis server 250 may provide the analysis results to the user terminal 220.

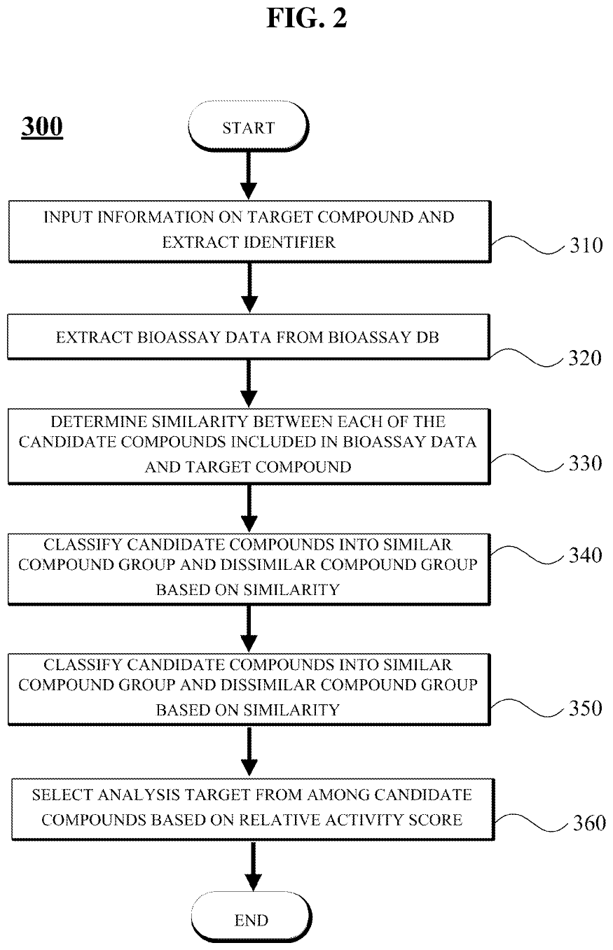

FIG. 2 is a flowchart for a process 300 of extracting drug information based on bioactivity data.

The analysis apparatus may receive the information on the target compound and extract an identifier for the target compound (310). The target compound may be a compound known to have activity with relation to at least one specific target protein. Alternatively, the target compound may be a compound whose specific activity is unknown.

The analysis apparatus extracts bioassay data from the bioassay DB (320). The analysis apparatus may extract all pieces of bioassay data or some pieces of bioassay data from the bioassay DB.

The analysis apparatus determines the similarity between each of the candidate compounds included in the bioassay data and the target compound (330). The analysis apparatus may identify the target compound based on the identifier of the target compound to determine structural characteristics of the target compound. Also, the analysis apparatus may extract structural characteristics of each compound based on the information on the candidate compounds included in the bioassay data. A method of determining compound similarity will be described below.

The analysis apparatus classifies candidate compounds into a similar compound group (simply, a similar group) and a dissimilar compound group (simply, a dissimilar group) depending on whether the candidate compounds are similar to the target compound (340).

The analysis apparatus may determine whether each of the candidate compounds has activity with relation to a specific target protein. When the specific target protein is set, the analysis apparatus may check whether each candidate compound has activity with relation to the target protein in the bioassay data. Meanwhile, the specific target protein may be preset or information that the analysis apparatus receives from a user.

The analysis apparatus may calculate a predetermined score depending on whether at least one of the compounds included in each of the similar compound group and the dissimilar compound group is activated (350). This score may be determined based on the similarity to the target compound and the activity of the candidate compound. This score is referred to as a relative activity score (RAS). The RAS is a score for bioassay data to be currently analyzed or candidate compounds included in the bioassay data. The RAS may be represented by Equation 1 below.

$$RAS = {log}_{2} \frac{( \frac{HS}{HD} )}{( \frac{AS}{AD} )} \qquad [ Equation 1 ]$$

Here, HS denotes the number of compounds whose activity is confirmed in the similar compound group. HD denotes the number of compounds whose activity is confirmed in the dissimilar compound group. AS denotes the number of compounds belonging to the similar compound group. AD denotes the number of compounds belonging to the dissimilar compound group.

Meanwhile, the analysis apparatus may calculate the RAS by Equation 2 below.

$$RAS = {log}_{2} \frac{( \frac{HS + α}{HD + α} )}{( \frac{AS + 1}{AD + 1} )} \qquad [ Equation 2 ]$$

In Equation 2, α denotes a Laplace smoothing parameter. α has a value of 1 or less. α may be determined as a result of an experimental evaluation. For example, α=0.001.

The analysis apparatus may select an analysis target from among the candidate compounds based on the RAS (360). The analysis apparatus may select an analysis target from among the candidate compounds with the RAS that is greater than or equal to a threshold value. For example, the analysis apparatus may select, as the analysis target, all or some compounds, which are activated, from among the candidate compounds. Alternatively, the analysis apparatus may select, as the analysis target, all or some compounds whose activity is confirmed from among the candidate compounds. Alternatively, the analysis apparatus may select, as the analysis target, all or some compounds, which belong to the similar compound group and are activated, from among the candidate compounds.

Furthermore, the analysis apparatus may select, as the analysis target, the bioassay data itself for candidate compounds having RAS greater than or equal to a threshold value. In this case, the analysis apparatus uses not only the compounds but also all pieces of the bioassay data as data for drug discovery.

Although not illustrated in FIG. 2, the analysis apparatus may store compounds or bioassay data selected as the analysis target in a discovery library DB.

When the target compound is a substance for which the specific medicinal efficacy is known, the analysis apparatus may predict that the analysis target selected based on the target compound also has the same medicinal efficacy as the target compound or an effect acting on a related mechanism.

Furthermore, when the specific activity or use of the target compound is unknown, the analysis apparatus may predict the activity or use of the target compound based on the analysis target that is selected based on the target compound. For example, the analysis apparatus may identify compound A having activity among the analysis targets and predict that the target compound will also have activity on the same target protein as the compound A. Alternatively, the analysis apparatus may identify compound B, which belongs to a similar compound group and has specific activity, among the analysis targets, and predict that the target compound will also have activity on the same target protein as the compound B.

Accordingly, the drug information extracted by the analysis apparatus may be diverse, such as a candidate list of a specific drug, a candidate list having activity associated with a specific compound, and a target of a specific compound.

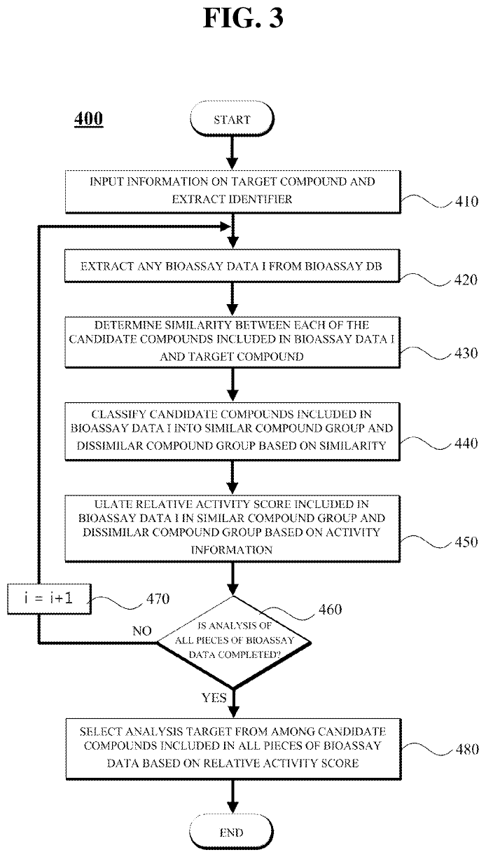

Meanwhile, the analysis apparatus may initially receive information on a plurality of target compounds (compound sets). In this case, the analysis apparatus may perform processes 320 to 360 on each of the plurality of target compounds in parallel or sequentially. FIG. 3 is another example of a flowchart of a process 400 of extracting drug information based on bioactivity data.

The analysis apparatus may receive the information on the target compound and extract an identifier for the target compound (410). The target compound may be a compound known to have activity with relation to at least one specific target protein. Alternatively, the target compound may be a compound whose specific activity is unknown.

The analysis apparatus extracts bioassay data from the bioassay DB (420). When there is a large amount of bioassay data in the bioassay DB, the analysis apparatus may extract some pieces of bioassay data in consideration of the performance of the analysis apparatus. For example, the analysis apparatus may extract one or more pieces of bioassay data. It is assumed that the analysis apparatus randomly extracts some pieces of bioassay data i from the bioassay DB. i represents one or a predetermined number of pieces of bioassay data. That is, i represents a unit of bioassay data that the analysis apparatus analyzes at once.

The analysis apparatus determines similarity between each of the candidate compounds included in the bioassay data i and the target compound (430). The analysis apparatus may identify the target compound based on the identifier of the target compound to determine structural characteristics of the target compound. Also, the analysis apparatus may extract structural characteristics of each compound based on the information on the candidate compounds included in the bioassay data. A method of determining compound similarity will be described below.

The analysis apparatus classifies the candidate compounds included in the bioassay data i into a similar compound group (simply, a similar group) and a dissimilar compound group (simply, a dissimilar group) depending on whether the candidate compounds are similar to the target compound (440).

The analysis apparatus may determine whether each of the candidate compounds has activity with relation to a specific target protein. When the specific target protein is set, the analysis apparatus may check whether each candidate compound has activity with relation to the target protein in the bioassay data. Meanwhile, the specific target protein may be preset or information that the analysis apparatus receives from a user.

The analysis apparatus may calculate a predetermined score based on the activity of at least one of the compounds included in each of the similar group and the dissimilar group of the bioassay data i (450). This score may be determined based on the similarity to the target compound and the activity of the candidate compound. The RAS is a score for bioassay data to be currently analyzed or the candidate compounds included in the bioassay data. The RAS may be represented by Equation 3 below.

$${RAS}_{i} = {log}_{2} \frac{( \frac{{HS}_{i} + α}{{HD}_{i} + α} )}{( \frac{{AS}_{i} + 1}{{AD}_{i} + 1} )} \qquad [ Equation 3 ]$$

i denotes i^{th} bioassay data.

The analysis apparatus confirms whether the analysis of all n pieces of bioassay data is completed (460). When the analysis of all n pieces of bioassay data is not completed (NO of 460), the analysis apparatus extracts the next bioassay data and repeats the processes 420 to 450. FIG. 3 illustrates the next bioassay data as i+1^{th} data (470).

When analysis of n pieces of bioassay data is completed (YES in 460), the analysis apparatus may calculate a final RAS as a result of analyzing all pieces of bioassay data. The final RAS may be represented by Equation 4 below. The analysis apparatus may determine the final RAS by averaging the total sum of RASs calculated for all the pieces of bioassay data based on the specific analysis unit i.

$$RAS = \frac{1}{n} {\sum }_{i = 1}^{n} {log}_{2} \frac{( \frac{{HS}_{i} + α}{{HD}_{i} + α} )}{( \frac{{AS}_{i} + 1}{{AD}_{i} + 1} )} \qquad [ Equation 4 ]$$

n denotes the number of times the analysis apparatus extracts bioassay data. When the analysis apparatus extracts one piece of bioassay data at a time, n may be the number of all pieces of bioassay data. i denotes i^{th} bioassay data.

The analysis apparatus may select an analysis target from among the candidate compounds based on the RAS (480). For example, the analysis apparatus may select, as the analysis target, all or some compounds, which are activated, from among the candidate compounds. Alternatively, the analysis apparatus may select, as the analysis target, all or some compounds whose activity is confirmed from among the candidate compounds. Alternatively, the analysis apparatus may select, as the analysis target, all or some compounds, which belong to the similar compound group and are activated, from among the candidate compounds.

Furthermore, the analysis apparatus may select, as the analysis target, the bioassay data itself for candidate compounds having RAS greater than or equal to a threshold value. In this case, the analysis apparatus uses not only the compounds but also all of the pieces of bioassay data as data for drug discovery.

Although not illustrated in FIG. 3, the analysis apparatus may store compounds or bioassay data selected as the analysis target in a discovery library DB. When the target compound is a substance for which the specific medicinal efficacy is known, the analysis apparatus may predict that the analysis target selected based on the target compound also has the same medicinal efficacy as the target compound or an effect acting on a related mechanism.

Furthermore, when the specific activity or use of the target compound is unknown, the analysis apparatus may predict the activity or use of the target compound based on the analysis target that is selected based on the target compound. For example, the analysis apparatus may identify compound A having activity among the analysis targets and predict that the target compound will also have activity on the same target protein as the compound A. Alternatively, the analysis apparatus may identify compound B, which belongs to a similar compound group and has specific activity, among the analysis targets, and predict that the target compound will also have activity on the same target protein as the compound B.

Accordingly, the drug information extracted by the analysis apparatus may be diverse, such as a candidate list of a specific drug, a candidate list having activity associated with a specific compound, and a target of a specific compound.

Meanwhile, the analysis apparatus may initially receive information on a plurality of target compounds (compound sets). In this case, the analysis apparatus may perform processes 420 to 480 on each of the plurality of target compounds in parallel or sequentially.

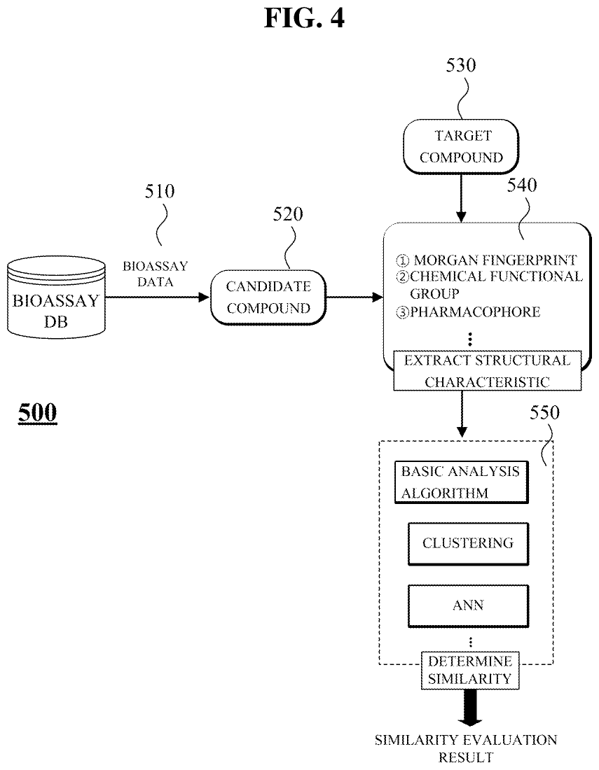

FIG. 4 is an example of a process of determining compound similarity (500).

The analysis apparatus receives information on a target compound (510). As described above, the analysis apparatus may define the target compound as an identifier of the target compound. The analysis apparatus extracts the bioassay data from the bioassay DB (510). The analysis apparatus identifies candidate compounds included in the extracted bioassay data (520). In this case, the candidate compound may also be represented as a specific identifier.

When the analysis apparatus identifies a target compound and/or a candidate compound as an identifier, the analysis apparatus needs to have structural information matching the identifier. The structure information may be represented in various types or formats. For example, the structural information may be represented in any one of types such as MOL, SDF, SMILES, InChI, and numerical vector. In this case, the analysis apparatus may extract the structure information of the compound indicated by the corresponding identifier from the table storing the structure information based on the identifier of the target compound and/or the candidate compound.

The analysis apparatus may receive the structural information of the target compound (510). In addition, the analysis apparatus may extract the structural information of the candidate compound from the bioassay data (530).

The analysis apparatus may evaluate whether the target compound and the candidate compound are similar based on at least one of various pieces of information. Similarity evaluation criteria may include a fingerprint, a chemical functional group, a pharmacophore and the like. The analysis apparatus first extracts the similarity evaluation criteria (structural characteristics) for each of the target compound and the candidate compound (540).

The analysis apparatus may evaluate the similarity between the target compound and each of the candidate compounds based on the structural characteristics (550).

The description below will be given based on the fingerprint. The analysis apparatus may generate the fingerprint based on the structural characteristics or physicochemical characteristics of the compound. The analysis apparatus may convert a SMILES format representing the compound structure into a vector value referred to as Morgan fingerprints. The analysis apparatus may generate the fingerprint, such as ECFP, for the target compound and each of the candidate compounds. The analysis apparatus evaluates the similarity of the Morgan fingerprints of the target compound and the candidate compound. The analysis apparatus may evaluate the similarity between the target compound and the candidate compound by calculating a Tanimoto coefficient or the like. The analysis apparatus may determine whether the target compound and the candidate compound are similar based on a preset threshold value. For example, the analysis apparatus may evaluate that the target compound and the candidate compound are similar when the Tanimoto coefficient is greater than or equal to a threshold value. Through this process, the analysis apparatus can classify candidate compounds into the similar compound group and the dissimilar compound group.

The analysis apparatus can also evaluate the similarity between the target compound and the candidate compound based on the chemical functional group or the pharmacophore. The analysis apparatus may evaluate the similarity between the target compound and the candidate compound based on the identity of the chemical functional group and the position of the chemical functional group. Alternatively, the analysis apparatus may evaluate the similarity based on the network between the pharmacophores or the arrangement position in a three-dimensional space. The analysis apparatus may evaluate the similarity based on numerical vectors representing compounds' structural information. Meanwhile, the similarity between the target compound and the candidate compound may be analyzed through various techniques. The analysis apparatus may also evaluate the similarity between compounds using commercial applications. The analysis apparatus may also evaluate the similarity by using the clustering technique based on the structure information of the fingerprint, the functional group, or the pharmacophore. Furthermore, the analysis apparatus may analyze the similarity between compounds by inputting information (for example, a fingerprint) represented by a specific vector value to an artificial neural network (ANN).

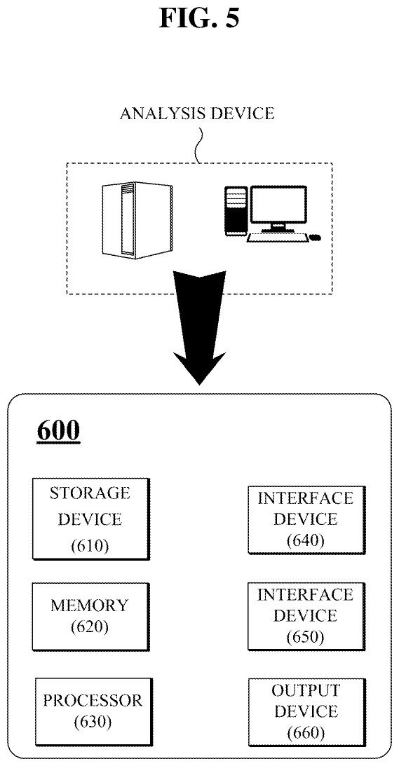

FIG. 5 is an example of an analysis apparatus 600 for discovery a drug based on bioactivity data. The analysis apparatus 600 corresponds to the above-described analysis apparatuses (150 and 250 of FIG. 1). The analysis apparatus 600 may be physically implemented in various forms. For example, the analysis apparatus 600 may have the form of a computer device such as a PC, a server of a network, a chipset dedicated to data processing, and the like.

The analysis apparatus 600 may include a storage device 610, a memory 620, a processor 630, an interface device 640, a communication device 650, and an output device 660.

The storage device 610 may store information on the target compound input by the user.

The storage device 610 may store a table that matches the compound identifier and the structure information.

The storage device 610 may store the bioassay data extracted from the bioassay DB.

The storage device 610 may store instructions or program code for a process of discovery a drug in the same manner as described above.

The storage device 610 may store information on a specific candidate compound or bioassay data on a specific candidate compound which is the analysis result.

The memory 620 may store data and information generated while the analysis apparatus 600 searches for a drug.

The interface device 640 is a device that receives predetermined commands and data from an external source. The interface device 640 may receive the information on the target compound from a physically connected input device or an external storage device. The interface device 640 may receive the input on the bioassay data from a physically connected input device or an external storage device. The interface device 640 may be referred to as an input device as a configuration for receiving predetermined information from a user or other physical objects.

The communication device 650 has a configuration for receiving and transmitting predetermined information through a wired or wireless network. The communication device 650 may receive the information on the target compound from an external object. The communication device 650 may receive the bioassay data from the bioassay DB. In addition, the communication device 650 may receive instructions or information required for the process of discovery a drug. The communication device 650 may transmit, to the discovery library DB, the information on the specific candidate compound or the bioassay data on the specific candidate compound which is the analysis result. Alternatively, the communication device 650 may transmit the analysis result to the user terminal.

The output device 660 is a device that outputs predetermined information. The output device 660 may output an interface necessary for a data processing process, an analysis result, and the like. The output device 660 may output a drug discovery result.

The processor 630 may screen a drug candidate related to the target compound using the instructions or program codes stored in the storage device 610.

The processor 630 may extract the identifier of the target compound and/or the candidate compound.

The processor 630 may extract the structural information of the target compound and/or the candidate compound based on the identifier of the target compound and/or the candidate compound.

The processor 630 may evaluate the similarity between the candidate compounds included in the bioassay data and the target compound. The processor 630 may extract the structural characteristics based on the structural information of the target compound and the candidate compound. The structural property may be at least one of characteristics groups including the fingerprint, the chemical functional group, and the pharmacophore. The processor 630 may evaluate whether the target compound and the candidate compound are similar by using at least one of various methodologies based on the structural characteristics.

The processor 630 classifies the candidate compounds into the similar compound group and the dissimilar compound group depending on whether the target compound and the candidate compound are similar.

The processor 630 may identify specific activity for the candidate compounds. The processor 630 may determine whether the specific candidate compound has activity with relation to the target protein based on quantitative information included in the bioassay data. The processor 630 may determine that the specific candidate compound has activity when the quantitative information included in the bioassay data is greater than or equal to a threshold value.

The target protein to be evaluated for activity may be a preset value. Alternatively, the target protein may be information input or received through the interface device 640 or the communication device 650.

The processor 630 may be a device such as a CPU, an AP, or a chip in which a program is embedded.

The processor 630 may calculate the RAS for the candidate compound included in the bioassay data. The RAS calculation has been described with reference to FIGS. 2 and 3. It is assumed that the calculation device 630 uses Equation 1. The variables used in Equation 1 may be summarized as shown in Table 1 below. In Table 1, the identifier means the identifier of the target compound.

TABLE 1

| | Identifier Similar | Identifier Dissimilar |
|---|---|---|
| | Compound (S) | Compound (D) |
| Hit Compound (H) | HS | HD |
| All Compounds (A) | AS | AD |

For example, the total number of compound sets is 6,600, the number of compounds similar to input compounds identified through a identification module is 200, and the number of compounds identified as the activity among the total of 6,600 compound sets is 300. When the number of compounds similar to the input compounds identified through the identification module and the similarity calculation module among the compound sets is 100, it is expressed as shown in Table 2 below.

TABLE 2

| | Identifier Similar | Identifier Dissimilar |
|---|---|---|
| | Compound (S) | Compound (D) |
| Hit compound (H) | 100 | 200 |
| All Compounds (A) | 200 | 6400 |

In this case, the analysis apparatus may calculate the RAS for the bioassay data or the candidate compounds included in the corresponding bioassay data as log₂(64)=6. When the threshold value is 5, the analysis apparatus may select the corresponding bioassay data or the candidate compounds included in the bioassay data as the analysis target. The analysis apparatus may store the corresponding bioassay data or at least some of candidate compounds included in the bioassay data in the discovery library DB. Alternatively, the analysis apparatus may predict the target protein of the candidate compounds belonging to the similar compound group as the target of the target compound.

Hereinafter, the experimental verification results for the above-described method of discovery a drug will be described.

In order to confirm the performance of a receiver-operating characteristic (ROC) curve specific prediction method, a curve with a true positive rate, that is, sensitivity, as a Y axis, and a false positive rate (1-specificity) as an X axis is indicated. An area under curve (AUC) value means an area under a curve in the ROC curve, and a large AUC value means that the validity or accuracy of the verification target is high.

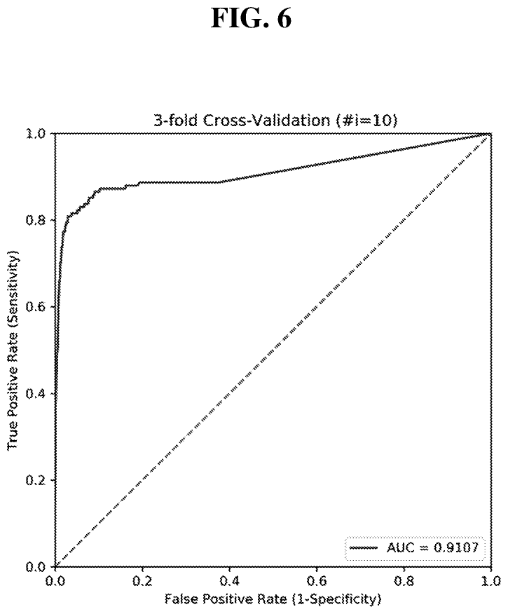

FIG. 6 is an example of experimental results according to the present embodiment.

As the bioassay DB, the bioassay DB (https://pubchem.ncbi.nlm.nih.gov) provided by the American Institute of Health was used. The target protein of the compound to be confirmed was set to “glutathione S-transferase theta 1, GSTT1 [Homo sapiens].” Researchers input a set of hit compounds known to the target protein as the target compound. Thereafter, researchers calculated the RAS calculated through the above-described analysis process for the bioassay data. To construct the discovery library, the process of selecting and analyzing the bioassay data was repeated 16,000 times or more, and the RASs for each bioassay were summed for the hit compound. A list of compounds was obtained by sorting the RASs in ascending order in the discovery library calculated through this process. That is, the list of compounds includes a set of compounds that are the hit compounds in the bioassay data and have a higher RAS. As the calculation result, it was confirmed that the AUC value was 0.9107 as illustrated in FIG. 6. In general, when the AUC value exceeds 0.7, the predictive performance is evaluated as high, and therefore, the above-described method of discovery a drug has been verified to have excellent predictive performance.

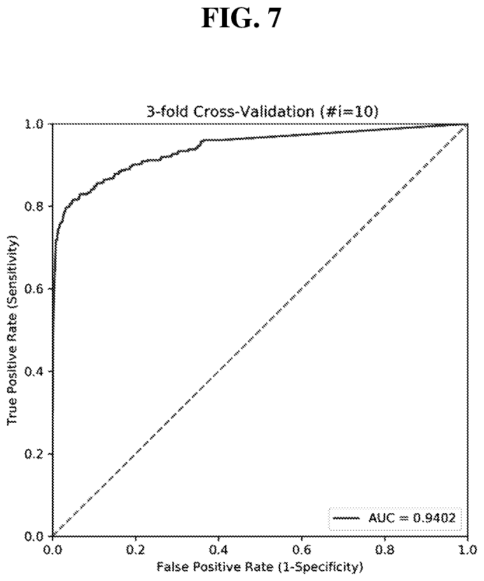

FIG. 7 is another example of the experimental results according to the present embodiment.

As the bioassay DB, the bioassay DB (https://pubchem.ncbi.nlm.nih.gov) provided by the American Institute of Health was used. The target protein of the compound to be confirmed was set as “Potassium Calcium-activated channel subfamily N member 2, KCNN2 protein [Homo sapiens].” Researchers set a set of hit compounds known to the target protein as the target compound. Thereafter, researchers calculated the RAS calculated through the above-described analysis process for the bioassay data. To construct the discovery library, the process of selecting and analyzing the bioassay data was repeated 16,000 times or more, and the RASs for each bioassay were summed for the hit compound. A list of compounds was obtained by sorting the RASs in ascending order in the discovery library calculated through this process. That is, the list of compounds includes a set of compounds that are the hit compounds in the bioassay data and have a higher RAS. As the calculation result, it was confirmed that the AUC value was 0.9077 as illustrated in FIG. 7. Therefore, the method of discovery a drug described above was proved to have the excellent predictive performance.

In addition, the method of extracting drug information, the method of discovery a drug, or the method of constructing a discovery library as described above may be implemented as a program (or application) including an executable algorithm that can be executed on a computer. The program may be stored and provided in a transitory or non-transitory computer readable medium.

The non-transitory computer readable medium is not a medium that stores data therein for a short time, such as a register, a cache, a memory, or the like but rather means a medium that semi-permanently stores data therein and is readable by a device. Specifically, various applications or programs described above may be stored and provided in a non-transitory computer readable medium such as a compact disk (CD), a digital video disk (DVD), a hard disk, a Blu-ray disk, a universal serial bus (USB), a memory card, a read-only memory (ROM), a programmable read only memory (PROM), an erasable PROM (EPROM), an electrically EPROM (EEPROM), or a flash memory.

The transitory readable medium means various random access memories (RAMs) such as a static RAM (SRAM), a dynamic RAM (DRAM), a synchronous DRAM (SDRAM), a double data rate SDRAM (DDR SDRAM), an enhanced SDRAM (ESDRAM), a synclink DRAM (SLDRAM), and a direct rambus RAM (DRRAM).

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A method of extracting drug information based on bioactivity data, comprising:
receiving, by an analysis apparatus, information on a target compound;
extracting, by the analysis apparatus, bioassay data from a bioassay database;
classifying, by the analysis apparatus, a plurality of candidate compounds included in the bioassay data into a similar compound group and a dissimilar compound group based on similarity to the target compound;
calculating, by the analysis apparatus, a relative activity score (RAS) based on activity information on compounds belonging to the similar compound group and the dissimilar compound group; and
selecting, by the analysis apparatus, at least some of the plurality of candidate compounds included in the bioassay data as an analysis target based on the RAS.

2. The method of claim 1, wherein the analysis apparatus predicts a target protein, in which the at least some compounds have activity, as a target of the target compound.

3. The method of claim 1, wherein the analysis apparatus evaluates the similarity between the target compound and each of the plurality of candidate compounds based on structural characteristics, and
the structural characteristics include at least one of characteristic groups including a fingerprint, a chemical functional group, a pharmacophore and a numeric vector.

4. The method of claim 1, wherein the RAS is calculated by an Equation below:
$$RAS = {log}_{2} \frac{( \frac{HS}{HD} )}{( \frac{AS}{AD} )} ,$$
wherein, HS denotes the number of compounds whose activity is confirmed in the similar compound group, HD denotes the number of compounds whose activity is confirmed in the dissimilar compound group, AS denotes the number of compounds belonging to the similar compound group, and AD denotes the number of compounds belonging to the dissimilar compound group.

5. The method of claim 1, wherein the RAS is calculated by an Equation below:
$$RAS = {log}_{2} \frac{( \frac{HS + α}{HD + α} )}{( \frac{AS + 1}{AD + 1} )} ,$$
wherein, HS denotes the number of compounds whose activity is confirmed in the similar compound group, HD denotes the number of compounds whose activity is confirmed in the dissimilar compound group, AS denotes the number of compounds belonging to the similar compound group, AD denotes the number of compounds belonging to the dissimilar compound group, and a denotes a Laplace smoothing parameter.

6. The method of claim 1, wherein the analysis apparatus repeatedly extracts at least one piece of bioassay data from the bioassay database without overlapping and calculates the RAS while classifying the similar compound group and the dissimilar compound group for each of the at least one piece of bioassay data.

7. The method of claim 6, wherein the RAS is calculated by an Equation below:
$$RAS = \frac{1}{n} {\sum }_{i = 1}^{n} {log}_{2} \frac{( \frac{{HS}_{i} + α}{{HD}_{i} + α} )}{( \frac{{AS}_{i} + 1}{{AD}_{i} + 1} )} ,$$
wherein, n denotes the number of times the bioassay data is extracted, i denotes i^{th} bioassay data, HS denotes the number of compounds whose activity is confirmed in the similar compound group, HD denotes the number of compounds whose activity is confirmed in the dissimilar compound group, AS denotes the number of compounds belonging to the similar compound group, AD denotes the number of compounds belonging to the dissimilar compound group, and a denotes a Laplace smoothing parameter.

8. The method of claim 1, wherein the analysis apparatus selects, as the analysis target, at least one compound belonging to a compound group that includes at least one compound whose activity is confirmed among the plurality of candidate compounds and at least one compound whose activity is confirmed in the similar compound group.

9. The method of claim 1, wherein the analysis apparatus calculates the RAS for each of the plurality of pieces of bioassay data, sums respective RASs for the compounds included in the plurality of pieces of bioassay data, and selects at least some of the compounds based on the summed RAS.

10. A method of constructing a drug discovery library based on bioactivity data, comprising:
receiving, by an analysis apparatus, information on a target compound;
extracting, by the analysis apparatus, bioassay data from a bioassay database;
classifying, by the analysis apparatus, a plurality of candidate compounds included in the bioassay data into a similar compound group and a dissimilar compound group based on similarity to the target compound;
calculating, by the analysis apparatus, a relative activity score (RAS) based on activity information on whether each of the compounds belonging to the similar compound group and the dissimilar compound group and a target protein are activated; and
selecting, by the analysis apparatus, the bioassay data as library data for drug substance research when the RAS is greater than or equal to a threshold value.

11. An analysis apparatus for discovery a drug based on bioactivity data, comprising:
an input device configured to receive information on a target compound;
a communication device configured to receive specific bioassay data from a bioassay database;
a storage device configured to store an instruction for discovery a drug candidate substance based on structural information and activity information of compounds; and
a processor configured to evaluate similarity between candidate compounds included in the bioassay data and the target compound, classify the candidate compounds into a similar compound group and a dissimilar compound group based on the similarity, calculate a relative activity score (RAS) based on activity information on the compounds belonging to the similar compound group and the dissimilar compound group, and select at least some of the candidate compounds as a drug candidate substance based on the RAS.

12. The analysis apparatus of claim 11, wherein the analysis apparatus evaluates the similarity between the target compound and each of the candidate compounds based on structural characteristics, and
the structural characteristics include at least one of characteristic groups including a fingerprint, a chemical functional group, and a pharmacophore.

13. The analysis apparatus of claim 11, wherein the RAS is calculated by an Equation below:
$$RAS = {log}_{2} \frac{( \frac{HS}{HD} )}{( \frac{AS}{AD} )} ,$$
wherein, HS denotes the number of compounds whose activity is confirmed in the similar compound group, HD denotes the number of compounds whose activity is confirmed in the dissimilar compound group, AS denotes the number of compounds belonging to the similar compound group, and AD denotes the number of compounds belonging to the dissimilar compound group.

14. The analysis apparatus of claim 11, wherein the RAS is calculated by an Equation below:
$$RAS = {log}_{2} \frac{( \frac{HS + α}{HD + α} )}{( \frac{AS + 1}{AD + 1} )} ,$$
wherein, HS denotes the number of compounds whose activity is confirmed in the similar compound group, HD denotes the number of compounds whose activity is confirmed in the dissimilar compound group, AS denotes the number of compounds belonging to the similar compound group, AD denotes the number of compounds belonging to the dissimilar compound group, and a denotes a Laplace smoothing parameter.

15. The analysis apparatus of claim 11, wherein the analysis apparatus sequentially extracts a plurality of pieces of bioassay data from the bioassay database and calculates the RAS while classifying the similar compound group and the dissimilar compound group for each of the plurality of pieces of bioassay data.

16. The analysis apparatus of claim 15, wherein the RAS is calculated by the following Equation:
$$RAS = \frac{1}{n} {\sum }_{i = 1}^{n} {log}_{2} \frac{( \frac{{HS}_{i} + α}{{HD}_{i} + α} )}{( \frac{{AS}_{i} + 1}{{AD}_{i} + 1} )} ,$$
wherein, n denotes the number of bioassay data, i denotes i^{th} bioassay data, HS denotes the number of compounds whose activity is confirmed in the similar compound group, HD denotes the number of compounds whose activity is confirmed in the dissimilar compound group, AS denotes the number of compounds belonging to the similar compound group, AD denotes the number of compounds belonging to the dissimilar compound group, and a denotes a Laplace smoothing parameter.
